(19) United States
(12) Patent Application Publication
(10) Pub. No.: US 2014/0308688 A1
(43) Pub. Date: Oct. 16, 2014

(54) HUMAN EMULATED RESPONSE WITH MICROFLUIDIC ENHANCED SYSTEMS

(71) Applicant: Research Triangle Institute, Research Triangle Park, NC (US)

(72) Inventors: Sonia Grego, Durham, NC (US); Brian Rhys Stoner, Chapel Hill, NC (US); Kristin Hedgepath Gilchrist, Durham, NC (US); Timothy Raymond Fennell, Raleigh, NC (US); Ann Pitruzzello, Durham, NC (US)

(21) Appl. No.: 14/362,942

(22) PCT Filed: Dec. 7, 2012
(86) PCT No.: PCT/US2012/068461

§ 371 Date: Jun. 5, 2014

Related U.S. Application Data
(60) Provisional application No. 61/568,201, filed on Dec. 8, 2011.

(51) Int. Cl. G01N 33/50

(52) U.S. Cl. CPC G01N 33/5014; G01N 33/5082
USPC 435/7.92; 435/297.1; 435/287.1; 435/297.2; 435/29

(57) ABSTRACT

A multiple flow-based microfluidic cell culture system that emulates mammalian physiology is provided. Tissue-mimicking cell cultures are connected by flow within a physiologically meaningful arrangement so that the pharmacokinetics of various agents to be tested in the system emulate in vivo conditions. The system includes at least two organ tissue modules, each organ tissue module including a first chamber containing an organ tissue cell, the first chamber including an inlet and an outlet for flow of an organ tissue cell-specific culture medium; a second chamber including an inlet and an outlet for flow of a blood material; and a semi-permeable membrane separating the first and second chambers. The flow of blood material through each organ tissue module is interconnected and the flow of tissue-cell specific culture medium is directed to a single organ tissue module.

### FIELD OF THE INVENTION

The present invention is directed to a human or other mammalian model system, and, in particular, a microfluidic model system that mimics mammalian physiology and pharmacokinetics.

### BACKGROUND OF THE INVENTION

The process of drug discovery and development requires a lengthy testing process beginning with the demonstration of pharmacological effects in high-throughput assays, experimental cell cultures, and animal models and ending with drug safety and efficacy studies in clinical trials. The high attrition rate of drug candidates is a major financial concern for the pharmaceutical industry, as drug failure may be identified only after substantial research and development resources are expended, and the current process results in a lengthy time to market for successful drugs. Drug failures can be attributed, in part, to a lack of effective pre-clinical models and assay systems.

One pre-clinical model historically used is that of the animal model. Animal models for preclinical drug development and toxicology, however, present issues of feasibility, human relevance and ethics. More recently, microfluidic platforms for cell culture that mimic human physiological response have been the object of intense research. Microfluidic technologies offer advantages over traditional microtiter cell culture plates by enabling control of the cell's microenvironment, including interaction with other cells, extracellular matrix, and soluble factors. These elements affect cellular phenotypes and more accurately mimic the in vivo tissue. A number of microfluidic perfusion systems have been developed for cell cultures, mostly aimed at developing new tools for drug and vaccine research with a focus on liver models. See Kim, L., Y. C. Toh, J. Voldman, and H. Yu, Lab Chip (2007) 7: 681-694; Wu, M.-H., S.-B. Huang, and G.-B. Lee, Lab Chip (2010) 10: 939-956. The use of a microfabricated structure enables replication of the cell microenvironments, which are designed at the microscale. Such a system is flexible enough to enable incorporation of multiple cell types for co-cultures as well as expose cells to well-controlled fluid flows thereby mimicking vascular and interstitial flow conditions occurring in vivo. Besides the development of tissue mimicking individual organs, efforts have been made to build systems with multiple organs connected by microchannels for investigation of drug toxicity and ADME (absorption, distribution, metabolism and extraction) simultaneously in multiple organs. Such an approach exposes the organs to the same solution to analyze drug metabolism and toxicity while taking into account the effect of secondary and tertiary metabolism. See U.S. Pat. No. 7,288,405 and U.S. Pat. No. 7,670,797.

A common limitation of prior approaches is the need for a common optimized culture medium to provide nutrients to all cells of the multiple-organ platform, which significantly limits the types of cells that can be used. Such an approach is adequate for immortalized cell lines, but immortalized cells do not model in vivo cellular physiology as accurately as do primary cells, which require specialized media for proper nutrition, differentiation, and expression. Other systems describe organs as cell cultures in compartments separated by membranes from the common interconnecting flowing medium, but do not provide specific (local) medium to each cell culture nor means to refresh such medium over time. Thus, there remains a need for a platform with microfluidic systems that provides a more accurate model of the human physiology.

### SUMMARY OF THE INVENTION

The present invention provides a system with multiple cell cultures that emulate organ tissue, each cell culture featuring multiple flow pathways. Tissue-mimicking cell cultures are connected in a physiologically meaningful arrangement by a common blood-like flow path, so that the pharmacokinetics of compounds or agents can be tested in a system that emulates in vivo conditions.

According to one aspect of the invention, a microfluidic system is provided. The system includes at least two organ tissue modules. According to one embodiment, the system includes two or more organ tissue modules corresponding to one or more of a liver, kidney, bone marrow, heart, brain or blood-brain barrier, or lung. The system can be scalable and capable of incorporating various other organ modules. Each organ tissue module can include a first microfluidic chamber including at least one organ tissue cell. The first chamber includes at least one inlet and at least one outlet for flow of an organ tissue cell-specific culture medium. Each organ tissue module also includes a second microfluidic chamber. The second chamber includes at least one inlet and at least one outlet for flow of a blood-mimicking material and a semi-permeable membrane separating the first and second chamber. In some embodiments, the second chamber includes endothelial cells.

The membrane enables interaction or diffusion between the first and second chamber of each organ tissue module. The membrane is typically a porous polymeric material (e.g., a nanoporous polymer) such that the cells do not cross the membrane, but cell secretions, proteins and other molecules can cross the membrane. The flow of blood material through each organ tissue module (also referred to as vascular flow) can be interconnected such that the blood material circulates through a plurality of organ tissue modules within the microfluidic system, and the flow of tissue-cell specific culture medium can be directed to a single organ tissue module such that the flow of tissue cell-specific culture medium to each organ tissue module is separated from the flow of tissue cell-specific culture medium to at least one other organ tissue module. Each organ tissue module typically includes a pump operably positioned to move the cell-specific culture medium through the first chamber.

In one embodiment, the system includes a pneumatic backplane pneumatically connected to each organ tissue module that includes air channels connected to a source of vacuum and a source of positive air pressure for pneumatic operation of at least one medium pump. The pneumatic backplane typically includes at least one electrical cable to enable electrical read-out of cellular activity.

The system can also include a fluidic backplane in fluid communication with each organ tissue module and adapted to connect each second chamber of each organ tissue module. The fluidic backplane typically includes at least one channel for vascular flow, at least one blood material pump, and a reservoir for blood material to enable blood material flow to each tissue module.

In certain embodiments, the system can be adapted to continuously re-circulate blood-like medium through a common fluidic circuit that connects each organ tissue module. The flow through each organ tissue module can be adapted to pharmacokinetically mimic blood flow in a human. The blood material can include cell culture medium, whole blood, or a composition comprising a component of whole blood, in particular plasma, proteins, platelets or red blood cells, or an oxygen-carrying blood substitute including hemoglobin-based oxygen carriers, crosslinked and polymerized hemoglobin, and perfluorocarbon-based oxygen carriers. Incorporating plasma and protein into the blood material flow enables evaluation of important factors to drug bioavailability such as plasma binding.

Various parameters of each organ tissue module or the overall system can be determined by physiologically based pharmacokinetics, such as organ tissue module size, residence time of cell culture media or blood material in each organ tissue module, or flow distribution of blood material through the microfluidic system. In certain embodiments, at least one organ tissue cell can be a primary cell. In one embodiment, the tissue cell can be a three dimensional cell construct. In one embodiment, the at least one organ tissue cell can be located at an air-liquid interface, on the membrane, or on an interior surface of the first chamber. Still further, the organ tissue cells can be part of a co-culture of multiple cell types, wherein all cell types are positioned on one side of the semi-permeable membrane or different cell types are positioned on each side of the semi-permeable membrane.

In one embodiment, the microfluidic system includes an organ tissue module adapted to mimic the heart, wherein the organ tissue module comprises a first microfluidic chamber comprising a plurality of cardiomyocytes therein and a second microfluidic chamber adapted to receive the flow of a blood material, the first microfluidic chamber separated from the second microfluidic chamber by the semi-permeable membrane, and further comprising a microelectrode array operatively positioned to make electrophysiological measurements of the cardiomyocytes.

In another embodiment, the above-noted organ tissue module adapted to mimic the heart is an integrated heart/lung organ tissue module further comprising a multi-chamber module adapted to mimic the air-liquid interface of a lung adjacent to the second microfluidic chamber and separated therefrom by a second semi-permeable membrane, the multi-chamber module comprising a first lung chamber adapted to receive a liquid culture medium and positioned adjacent to the second semi-permeable membrane and a second lung chamber comprising alveolar epithelial cells and adapted to receive a flow of air, the second lung chamber separated form the first lung chamber by a third semi-permeable membrane.

In a further embodiment, the microfluidic system includes one or more organ tissue modules adapted to mimic an organ selected from the group consisting of liver, kidney, and bone marrow, the organ tissue module comprising a first microfluidic chamber comprising a plurality of cells selected from the group consisting of liver cells, kidney cells, and bone marrow cells, and a second microfluidic chamber adapted to receive the flow of a blood material, the first microfluidic chamber separated from the second microfluidic chamber by the semi-permeable membrane. In certain embodiments, the system comprises an organic tissue module comprising liver cells, an organ tissue module comprising kidney cells, and an organ tissue module comprising bone marrow cells.

In certain embodiments, the microfluidic system includes an organ tissue module adapted to mimic the blood-brain barrier, wherein the organ tissue module comprises a first microfluidic chamber comprising a plurality of brain glial cells and a second microfluidic chamber adapted to receive the flow of a blood material and comprising a plurality of brain endothelial cells, the first microfluidic chamber separated from the second microfluidic chamber by the semi-permeable membrane. Typically, the brain glial cells and the brain endothelial cells are seeded on opposite sides of the semi-permeable membrane.

In one embodiment, each organ tissue module is in fluid communication with a fluidic backplane that defines a recirculating flow path for the flow of blood material such that the blood material can continuously circulate through each second microfluidic chamber of each organ tissue module, and each organ tissue module is removably connected to the fluidic backplane. Typically, each organ tissue module defines a separate flow path for the organ tissue cell-specific culture medium that is wholly contained within the organ tissue module. The flow path for the organ tissue cell-specific culture medium typically comprises a channel extending from a first reservoir on the organ tissue module, through the first microfluidic chamber, and to a second reservoir on the organ tissue module. The organ tissue modules typically also include a pump operably positioned to move the organ tissue cell-specific culture medium through the first microfluidic chamber, which is often pneumatically operated through connection to a pneumatic backplane that is pneumatically connected to each organ tissue module.

In one specific embodiment, the microfluidic system of the invention comprises the following:

a lung module comprising alveolar epithelial cells and adapted to mimic the air-liquid interface of a lung;

a heart module comprising cardiomyocytes; and

at least one of (i) a blood-brain barrier module comprising brain glial cells and brain endothelial cells; (ii) a liver module comprising liver cells; (iii) a kidney module comprising kidney cells; and (iv) a bone marrow module comprising bone marrow cells;

wherein the blood material flow through each organ tissue module is adapted to pharmacokinetically mimic blood flow in a human.

According to another aspect of the invention, a method of analyzing tissue response to an agent is provided. The method includes the steps of providing a microfluidic system, such as any of the system embodiments noted above, administering an agent to at least one cell of at least one organ tissue module, and evaluating any physiological response or injury to the at least one cell or to any organ tissue cells in any of the organ tissue modules. The agent can be a drug, toxin or pathogen. The agent can be administered in a variety of ways, such as by administering the agent to the flow of blood material, administering the agent to an organ tissue cell-specific culture medium of one or more of the organ tissue modules, or administering the agent to the air at the air-liquid interface of a lung module. Examples of methods of evaluating physiological response or injury include analysis of cell secretions from one or more organ tissue modules or optical imaging of one or more organ tissue modules.

### BRIEF DESCRIPTION OF THE DRAWINGS

FIG. 1 illustrates an integrated board according to one embodiment of the instant system.

FIG. 2(a) illustrates an organ tissue module according to one embodiment of the instant system.

FIG. 2(b) illustrates an exploded view of the organ tissue module of FIG. 2(a).

FIG. 3 illustrates the flow pattern of one embodiment of the present system whereby the flow emulates the human response to pulmonary exposure to a drug or other agent.

FIG. 4 illustrates the arrangement of organs according to a physiologically based pharmacokinetic description of the body according to one embodiment.

FIG. 5 illustrates a schematic of a multi-organ system that includes a cell culture area proportional to organ volume and physiological blood flow distribution according to one embodiment.

FIG. 6 illustrates an exemplary configuration of a cardiac module according to one embodiment.

FIG. 7 illustrates the integration of a cardiac module with a lung module with a cross-section view of a lung epithelial layer exposed to xenobiotics.

FIG. 8 illustrates an exemplary configuration of a liver module according to one embodiment.

FIG. 9 illustrates how a co-culture of brain endothelial cells is implemented in a blood-brain barrier module of the present invention.

### DETAILED DESCRIPTION OF THE INVENTION

As used herein, the term “fluid” refers to air, liquid, or a combination thereof.

As used herein, the term “fluidic” refers to system or apparatus adapted for transport of a fluid therethrough.

As used herein, the term “microfluidic” refers to a fluidic pathway that includes at least one dimension of less than one millimeter.

As used herein, the term “pathogen” refers to microorganism such as a virus, bacterium, prion, or fungus that may cause disease in a host organism.

As used herein, the term “agent” refers to any chemical or biological composition intended to elicit a response from the cells of the microfluidic system of the invention such as a drug, toxin, or pathogen.

As used herein, the term “organ” refers to a group of cells or tissues that perform a specific function or group of functions.

The present invention provides a microfluidic system that enables the studying and evaluation of cell function in vitro under conditions closely resembling those in vivo. Various parameters in the system can be closely controlled, allowing for the observation of cell response to known inputs for a given set of conditions. In certain embodiments, the system mimics important functional components of various organs. Thus, pharmaceuticals targeted for various organ or drug therapies can be developed and evaluated using the system of the present invention, and the toxicity of the drug to one or more of the organs represented can be evaluated. Exemplary drugs or therapies that can be tested include, but are not limited to, those set forth in U.S. Pat. No. 7,670,797, the disclosure of which is incorporated herein by reference. Drug efficacy and toxicity can be tested safely in vitro by evaluating organ cells for viability and markers of cell health and function after an organ representing a tissue mucosa permeable to the pharmaceutical has been exposed to the compound or after the pharmaceutical is added to the vascular flow and through the vascular flow is delivered to all organs. Absorption, distribution, metabolism and extraction functionality can be carried out by one or more of the cellular modules on the pharmaceutical under test, a level of information not available in traditional in vitro methods of drug testing. Drug candidates can be investigated by the platform of this invention early in the development process to identify those with potential efficacy in humans (to avoid development of drugs which are effective in animals but not in humans) as well as those with potential for toxicity.

### System Design

A. Overview

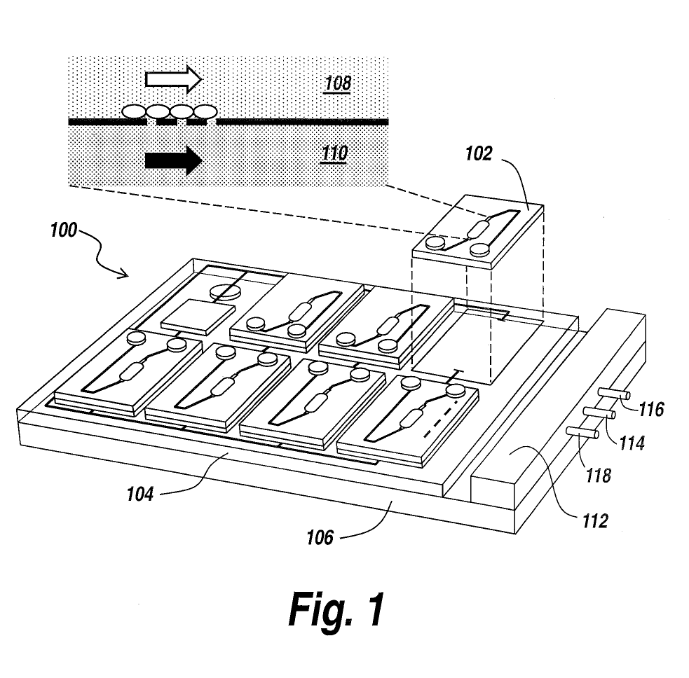

FIG. 1 illustrates one embodiment of the microfluidic system 100 of the present invention. Organ modules 102 are assembled on a fluidic backplane 104 which, in turn, is assembled on a pneumatic backplane 106. As illustrated in FIG. 1, the microfluidic system 100 includes various organ modules 102 such as, for example, a liver, kidney, bone marrow, heart, lung, and brain barrier module. The system 100 of the present invention may be fabricated in a modular and scalable fashion and additional organ modules 102 can be added to the system 100 as needed.

Each module 102 of the system 100 enables at least two fluidic pathways. One pathway allows flow of an organ tissue cell-specific culture medium 108 (e.g., functional medium) to a specific organ tissue module. A second pathway is the common fluidic pathway 110 that allows flow (e.g., vascular flow) of blood material throughout the entire system 100. Each module 102 typically contains on-board pneumatic valves, pump and reservoirs for medium refreshment in the functional cells compartment.

As noted above, an integrated board typically includes a fluidic backplane 104 and a pneumatic backplane 106. The fluidic backplane 104 includes an interconnected fluidic pathway between individual organ modules 102 and a pump and reservoir(s) for blood material. In one embodiment, the fluidic backplane 104 adopts the use of open wells for pipette-friendly culture media reservoirs. The fluidic backplane 104 includes an opening to enable connection from the modules 102 to the pneumatic backplane 106.

The pneumatic backplane 106 includes air channels for operating medium pumps. The air channels are connected via a manifold 112 to a single source of vacuum via vacuum connection 114 and single source of air pressure via air connection 116. The manifold 112 includes non-disposable components such as electrically controlled microvalves to independently drive the operation of the pumps. An electrical channel 118 mounted on the manifold 112 provides a conduit for the electrically controlled microvalves. A programmable interface can be implemented to control the microvalves. One embodiment of the system of the invention utilizes on-board valves and pumps on each module 102 that rely on a mechanism of elastic response of an elastomeric membrane fixed between two rigid layers on the backplane and deformed by air pressure.

The modules can be disconnected from the board for a variety of purposes, such as microscopic observation. Disconnection of the module stops fluid flow because the connections to the driving pneumatic channels are interrupted. Valves can be implemented to prevent leakage of media when the module is disconnected. In one embodiment, the various cell cultures can remain viable for at least one hour without media flow that provides oxygen and nutrients.

In a preferred embodiment, the system is three-dimensional. Certain three-dimensional systems are described, for example, in U.S. Pat. No. 6,455,311; U.S. Pat. No. 7,670,797, and U.S. Patent Publication No. 2011/0082563, the contents of which are each incorporated herein by reference for their detailed descriptions, figures and examples, which describe the structure and function of three-dimensional tissue engineered systems.

B. Organ Module

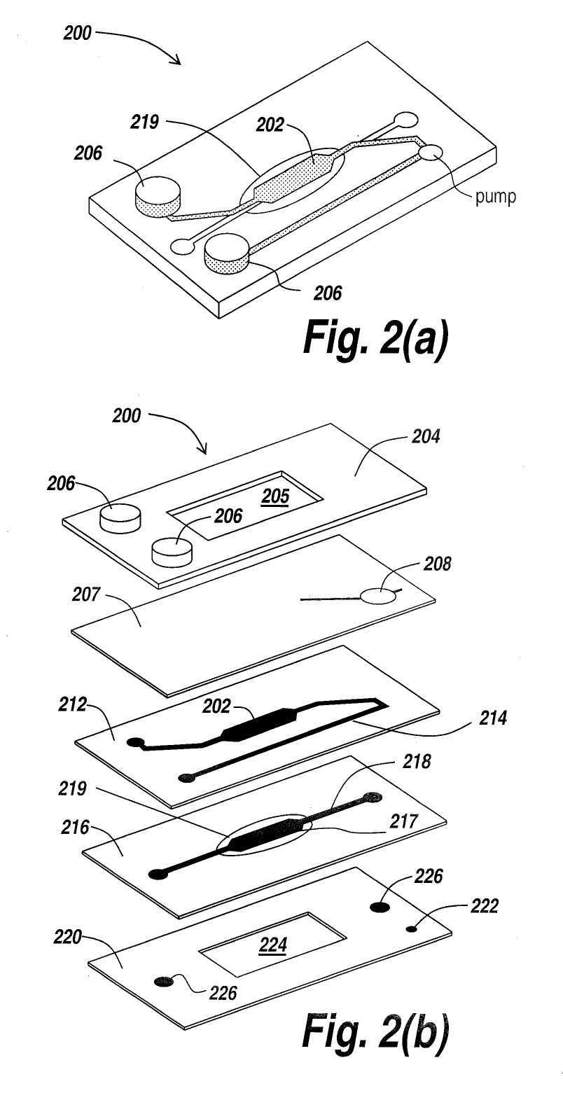

FIG. 2(a) illustrates an organ tissue module 200 according to one embodiment. FIG. 2(b) illustrates an exploded view of an organ tissue module 200. Referring to FIGS. 2(a) and 2(b), the system of the present invention includes at least one organ module 200 that includes tissue cells from at least one organ seeded within a first compartment 202 (typically microfluidic in size). The tissue module 200 includes a reservoir layer 204 containing reservoirs 206 (inlet and outlet) for medium which flows to the first compartment 202. The reservoir layer 204 can further include a top viewing window 205. In one embodiment, the system further includes a pneumatic layer 207 that includes a pneumatically actuated diaphragm pump 208 operating in aspiration to draw liquid medium from the respective reservoir 206 to the respective outlet. In one embodiment, the culture media each flow in a unidirectional manner.

The system further includes: (i) a tissue-specific fluidic layer 212 containing the fluid compartments or channels 214 (typically microfluidic in size) as well as the respective organ cells seeded within the first compartment 202; and (ii) a common fluidic layer 216 containing a second compartment 217 (typically microfluidic in size) and fluid compartments or channels 218 (typically microfluidic in size) that flow blood material to each module 200. The first compartment 202 and second compartment 217 are separated by a semi-permeable (e.g., nanoporous) membrane 219. A bottom viewing layer 220 includes at least one pneumatic connection 222 for the tissue fluidic channels 214, a viewing window 224, and at least one valving component 226, such as check valves, for preventing leakage of vascular medium when the module is disconnected from the board.

As noted above, each tissue module can include at least two compartments: (i) a first compartment containing the respective organ tissue cells that receives tissue specific functional cell culture media; and (ii) a second compartment that mimics vascular physiology. In one embodiment, the two compartments can be separated by a semi-permeable or nanoporous membrane. In one embodiment, the membrane can be selectively permeable to some ions and molecules, but not to other ions and molecules, depending upon physical or chemical properties of the molecule and the membrane. The membrane further allows for interaction or diffusion between two different media. In a preferred embodiment, the pore size of the membrane can be smaller than the cell diameters, thus, cells cannot pass through (i.e., a low permeability for mammalian cells), while low molecular weight nutrients and fluids can pass through (i.e. a high permeability for nutrients), thereby providing adequate cell-to-cell signaling because of diffusion across the membrane.

In a preferred embodiment, the individual organ modules are exposed to a re-circulating, common blood material or vascular flow during drug test. In such an embodiment, the blood material can be continuously re-circulated through a common fluidic circuit of each second compartment and is thereby connected to all organ modules, where the blood materials receives secretions from cellular processing of drug compounds from potentially any module and distributes the secretions to the entire system. In one embodiment, the blood material is whole blood or a blood surrogate such as a composition comprising a component of whole blood such as platelets or red blood cells, or a composition comprising an oxygen-carrying blood substitute such as hemoglobin-based oxygen carriers (HBOCs), including crosslinked and polymerized hemoglobin, and perfluorocarbon-based oxygen carriers (PFBOCs).

According to one embodiment, a bottom viewing window in the bottom layer can also be included for microscopy observation of the cell culture. A top viewing window in the reservoir layer can be included and is formed by suitably thinning the transparent polymer forming the module or by affixing a thin glass or glass coverslip over the cell culture region. The module can be detached from the integrated backplane for microscopy observation through the bottom viewing window.

Analysis of media effluent enables evaluation of each individual organ of the system. In one embodiment, the blood material optionally includes a non-disruptive tap and refill for continuous monitoring of effluent. In use, the system of the present invention allows for either batch or continuous flow sampling. Such sampling can be conducted by tapping into the primary flow channel in a manner so as not to disrupt concentrations and feeding the tapped flow to an external sensor for real-time analyses or collection for later measurement.

According to one embodiment, the system can include more than one organ. In one embodiment, the system includes six organ modules based on the role played in drug safety testing and toxicology studies. In such an embodiment, the six organ modules can include the lung, cardiac muscle, liver, brain, bone marrow, and kidney. In one embodiment, the lung represents the human airway compartment.

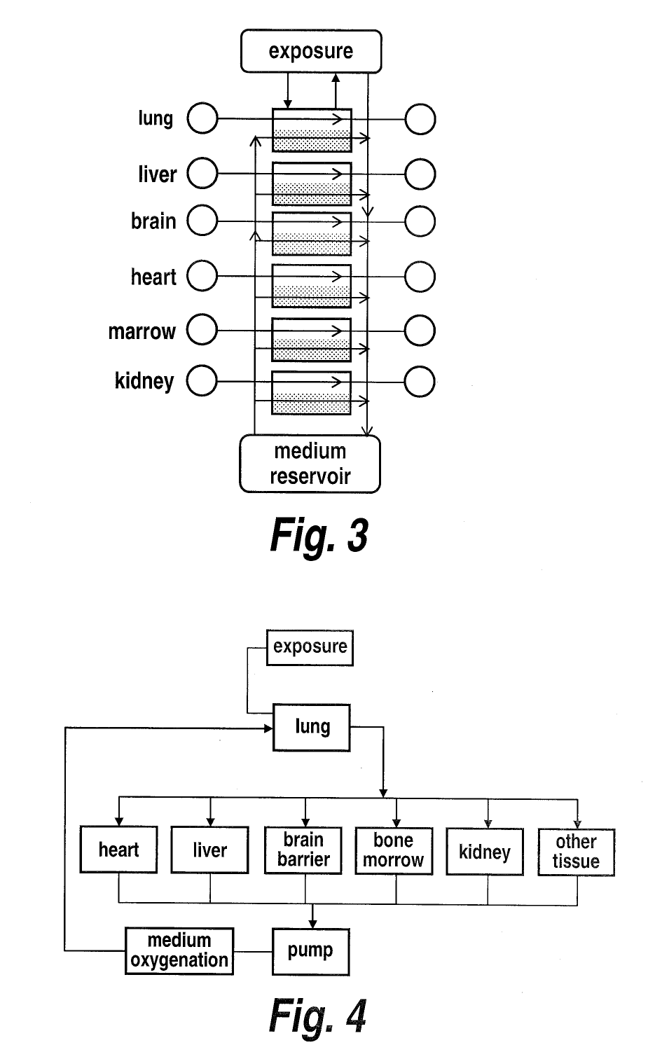

One embodiment of a system is illustrated in FIG. 3 that shows the flow pattern of a system emulating human response to exposure to a drug or other agent. As illustrated, each box represents an organ tissue module. The horizontal lines (connected to circles) represent each fluidic pathway that allows flow of an organ tissue cell-specific culture medium to a specific organ tissue module. A second pathway is a common fluidic pathway that allows flow of blood material (e.g., circulating medium) throughout the entire system. According to the embodiment of FIG. 3, exposure to a drug or other agent occurs in the lung thus allowing for analysis and evaluation of any physiological response or injury throughout the system. Preferably, the lung model includes cells grown at an air-liquid interface, so that the exposure can occur both in gas phase as well as through deposition of a small liquid amount on the air-exposed surface of the cell culture. Alternatively, the drug can be added to the blood material.

### Pharmacokinetics

In a preferred embodiment, the organ mimic modules and their fluidic connection arrangement are designed to emulate physiological conditions, including pharmacokinetic principles. Pharmacokinetic models are hypothetical structures that are used to describe the fate of a drug in a biological system following its administration. By providing a system of cell-culture based organ mimics designed according a pharmacokinetic model of the body, the predictive value and in vivo relevance of screening and toxicity assays is enhanced. In one embodiment, features, designs and validation of geometries based on a physiological-based pharmacokinetic (PBPK) model may include those set forth in U.S. Pat. No. 7,288,405, the contents of which are incorporated herein by reference.

One embodiment of an arrangement of the organs according to pharmacokinetic principles is shown in FIG. 4. The blood flow in the human body is such that the lung receives 100% of the cardiac output. The heart acts as a pump and receives substantially all of the flow. The heart muscle and the other organs each receive a fraction of the blood flow. Referring to FIG. 4, the lines between boxes (e.g., modules) indicate re-circulating medium (e.g., blood material). The lung receives about 100% of the cardiac output while the remaining organs are arranged in parallel. In one embodiment, the tissue of the cardiac module comprises heart muscle which receives typically about 4% of the flow. The cardiac module therefore connects in parallel with the other organ modules. The flow to the other organ modules can be scaled according to the approximate flow values according to the embodiment of Table 1. See also R. P. Brown, M. D. Delp, S. L. Lindstedt, L. R. Rhomberg, and R. P. Beliles, Physiological parameter values for physiologically based pharmacokinetic models, Toxicol Ind Health 13 (1997) 407-84. The cell culture area can be scaled to be proportional to the organ weight as illustrated in Table 1.

TABLE 1

| | Weight | Weight | Blood flow |
|---|---|---|---|
| | (% total | normalized | (% cardiac |
| Organ | body) | to liver | output) |
| Liver | 2.57 | 100% | 22.7 |
| Bone | 2.1 | 82% | 4.2 |
| marrow* | | | |
| Kidney | 0.44 | 17% | 17.5 |
| Lung | 0.76 | 30% | 100 |
| Heart | 0.47 | 18% | 4 |
| Brain | 2 | 78% | 11.4 |

*red and yellow marrow

Referring to FIG. 5, one lung compartment emulates the conducting airways (labeled “lung/bronchi” in figure) and one emulates the alveolar blood-oxygen-exchanging region (labeled “lung/alveoli” in figure). The illustrated percentages represent the relative blood flow to each organ module which mimics the human physiology. The cell culture in both the bronchial and alveolar modules represent functionality of permeability, active transport, and stress response to pathogen. The gas exchange (i.e., oxygenation) functionality for the vascular medium is typically carried out by non-cellular means (labeled “gas exchange” in FIG. 5; labeled “medium oxygenation” in FIG. 4).

According to one embodiment, the residence time of fluid in a tissue is emulated with respect to in vivo physiology. To achieve a time-dependent response, the system can include resistive components at each module and fluidic capacitors. In one embodiment, resistive and capacitive elements are included along the fluidic path to enable implementation of a time delay to thereby emulate different residence times of fluids into different organ compartments. In one embodiment, a fluidic capacitor can be obtained by bonding a deformable film over reservoirs placed in the system between fluidic channel resistors fabricated of a rigid material. (D. C. Leslie, C. J. Easley, E. Seker, J. M. Karlinsey, M. Utz, M. R. Begley, and J. P. Landers, Frequency-specific flow control in microfluidic circuits with passive elastomeric features, Nature Physics 5 (2009) 231-235).

### Materials and Fabrication

According to one embodiment, the semi-permeable membrane used in each organ module can be fabricated from at least one polymer. Polycarbonate, polyester, polyethylene terephthalate, polyethersulfone, polypropylene, and cellulose based membranes are exemplary membranes suitable for use in the invention. According to one embodiment, track-etched membranes can be used, which allow transport in the transverse direction to the membrane only and exhibit a well-defined pore size. In a preferred embodiment, a polyester membrane such as a Transwell™ membrane can be utilized because of advantageous properties of such membranes, such as biocompatibility, optical transparency, and permeability. According to one embodiment, the membrane is typically from about 1 micron to about 100 microns thick, with a preferred value of about 10 μm. Pore size typically ranges typically from about 0.1 μm to about 10 μm. In a preferred embodiment, pore size of the membrane is between about 0.2 μm and about 1 μm.

The compartments and remaining supporting structure of the system may be fabricated from at least one polymer such as polymethylmethacrylate (PMMA) or other acrylic polymers, polypropylene (PP), cyclic olefin copolymer (COC), polyethersulfone, polyvinyl chloride, polyester, polycarbonate, polystyrene, polydimethylsiloxane, polyethylene, or a fluoropolymer. In one embodiment, the system compartments and supporting structures are fabricated by a microfabrication approach such as, for example, wet etching, plasma dry etching, or soft lithography and micromolding or precision machining such as CNC (computer numerical control) precision, injection molding or embossing. In a preferred embodiment, polymer laminate technology is used, which combines laser patterning of polymer sheets designed for layered assemblies. Thin film adhesives, such as polyurethane, pressure sensitive adhesives or silicones are used to bond the layers.

In a preferred embodiment, the fabrication approach enables incorporation of elastomeric valves in the device. The valve can be obtained by designing a valve seat connected to air pressure on one side of a flexible elastomeric layer and a flow channel on the other side of the elastomer. Air pressure can be used to actuate the elastomer in and out of the valve seat to interrupt or enable the flow in the fluidic channel. A pump can be obtained by alternating operation of two valves connected on each side of the pneumatic-operated membrane. Elastomeric materials used to obtain fluidic-integrated pneumatic valves include, but are not limited to, polyurethane, silicone, acrylic polymers and polydimethylsiloxane.

Carcinoma-derived cell lines and immortalized cell lines can be utilized. In a preferred embodiment, cells utilized in the model system can be procured, dedicated, and quality verified from living tissue. For evaluating a particular drug therapy, cells from a specific organism such as, for example, a human may be obtained through medical procedures performed by medical professionals such as, for example, biopsy or harvest from a living donor, cell culture, or autopsy.

Module cell seeding and growth can occur independently for each module before insertion into the board since each cell type can exhibit different growth and differentiation time. The tissue of each module corresponds to an individual organ. Thus, the modules of the instant invention can include one or more types of functional, mesenchymal or parenchymal cells, such as smooth or skeletal muscle cells, myocytes (muscle stem cells), fibroblasts, chondrocytes, adipocytes, fibromyoblasts, ectodermal cells, including ductile and skin cells, hepatocytes, macrophages, kidney cells, cardiomyocytes, enterocytes, bronchial epithelial cells, alveolar epithelial cells, neurons, vascular endothelial cells, osteoblasts and other cells forming bone or cartilage, and hematopoietic cells. A stem cell includes, but is not limited to, embryonic stem cells, adult stem cells, neural stem cells, muscle stem cells, hematopoietic stem cells, mesenchymal stem cells, peripheral blood stem cells and cardiac stem cells. In a preferred embodiment, the stem cell is human. A stem cell can also be a pluripotent, multipotent or totipotent cell that can undergo self-renewing cell division to give rise to phenotypically and genotypically identical daughter cells for an indefinite time and can ultimately differentiate into at least one final cell type.

According to one embodiment, the system of the present invention can be used for the culturing of cells. The first compartment can be populated with cells of a single or of multiple types, which may be placed at distinct locations within the compartment or directly on a semi-permeable membrane. The relative placement and shape of the compartment, the cell location inside the compartment, and operational parameters such as medium compositions and pressures enable control over the microenvironment of the cultured cells and can allow for the administration of chemical, biological, mechanical, electrical, and biophysical signals to the cells. In one embodiment, the system of the present invention can be utilized to influence cell function and to facilitate culture of multiple cell types at distinct locations within the first compartment. A user may administer any combination of parameters, simultaneously or in time according to a specific scheme, in order to alter cell function in a desired fashion for a particular purpose. Cell enhancement or limitation of proliferation, the maintenance of stem cell pluripotency, or the differentiation of cells towards a specific phenotype can be altered and evaluated.

According to one embodiment, primary or stem-cell derived cells can be utilized to more accurately reproduce in vivo physiology. Primary cells and some stem cell derived cells are maintained through the use of a specialized culture medium for each tissue module.

### Individual Organ Modules

A. Heart

The system of the present invention may include a cardiac module. Cardiac toxicity has been a leading cause of drug-withdrawal from clinical studies and from the market for both cardiac and non-cardiac drug compounds. For example, QT prolongation, which indicates delayed ventricular repolarization, is associated with the potentially fatal arrhythmia torsades de pointes (TdP), which has been a common reason for withdrawal of promising drugs. (A. J. Camm, Clinical trial design to evaluate the effects on cardiac repolarization: current state of the art, Heart Rhythm. 2 (2005) S23-S29; C. Chiang, Drug-induced long QT syndrome, J. Med. Biol. Eng. 25 (2006) 107-113; P. J. Kannankeril, D. M. Roden, Drug-induced long QT and torsade de pointes: recent advances, Curr. Opin. Cardiol. 22 (2007) 39-43.) Thus, assessing risk for QT interval prolongation is a mandatory part of preclinical evaluation of all drugs in development. Long QT has most commonly been associated with loss of current through hERG potassium channels due to direct block of the ion channel by drugs or by inhibition of the plasma membrane expression of the channel protein. Long QT can also be caused by drug interaction with other ion channels besides hERG, or through interaction with multiple ion channels. Thus, hERG tests are necessary but not sufficient for accurate safety pharmacology. Besides electrophysiological effects, drugs are known to have additional toxic effects on the heart. Such toxic effects include drug-induced formation of reactive oxygen species, apoptosis, or altered molecular signaling. Thus, integration of an in vitro cardiac module along with other fluidic organ modules in the system of the present invention allows for a variety of drug studies, disease models, and toxicology studies.

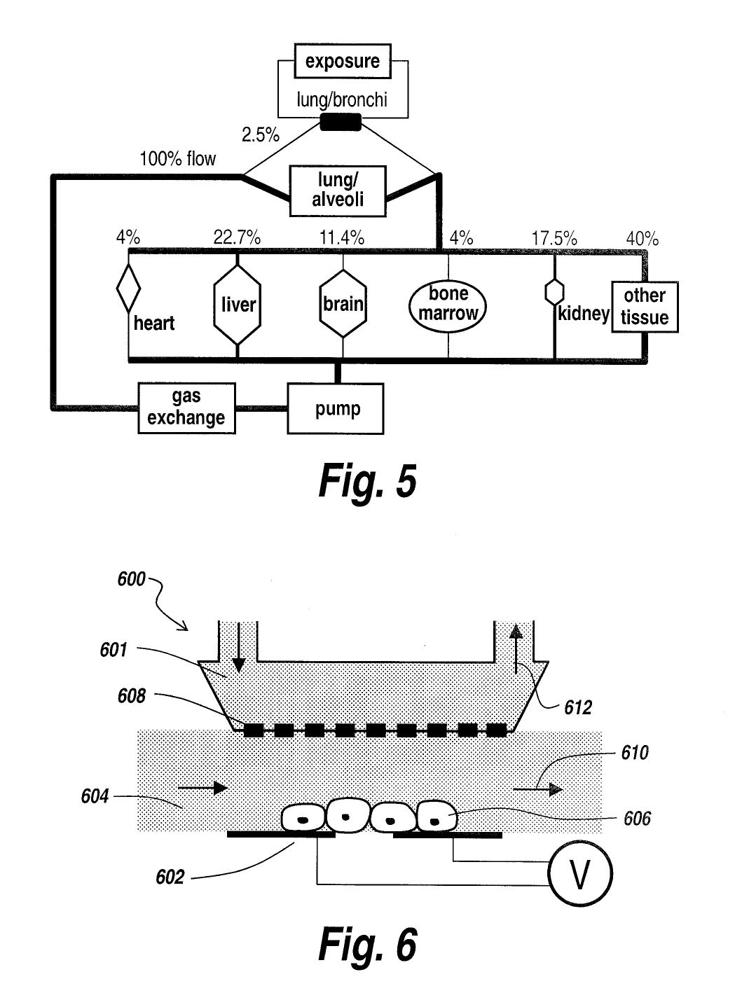

One embodiment of a cardiac module 600 is shown in FIG. 6. A microelectrode array 602 is embedded in the bottom wall of a compartment 604 containing cardiomyocytes 606, which can be cultured and grown in one or more layers on the microelectrode array. In one embodiment, the cardiomyocytes 606 are primary cells, cell lines, human embryonic stem cells or, preferably, human induced pluripotent stem cells. (H. Andersson et al., Assaying cardiac biomarkers for toxicity testing using biosensing and cardiomyocytes derived from human embryonic stem cells, J. Biotech. 150 (2010) 175-181; S. J. Kattman et al., Stem cells and their derivatives: A renaissance in cardiovascular translational research, J. Cardiovasc. Trans. Res. (2011) 4:66-72.) Human induced pluripotent stem cells derived from cardiomyocytes have the advantage of quantity availability and reproducibility versus primary cells. Additionally human induced pluripotent stem cells can be used to create population-specific or patient-specific cells or specific disease models. These cells have the potential of being a scalable and inexhaustible source for cardiac safety toxicology. (S. R. Braam, et al. Prediction of drug-induced cardiotoxicity using human embryonic stem cell-derived cardiomyocytes. Stem Cell Res. (2010) 4, 107-116.)

Electrophysiological testing of a drug candidate has historically been tested by a patch clamp which is low-throughput, high-cost, and does not replicate full cellular electrophysiology. Microelectrode arrays provide a non-invasive and long-term method to detect extracellular field potentials from cultured cells and from which arrhythmia and long QT can be assessed. Thus, the microelectrode array allows for continuous, direct electrophysiological measurements of the cardiac cells.

As with other modules of the present invention, and in reference to FIG. 6, a vascular compartment 601 and the compartment or chamber 604 containing cardiomyocytes 606 are separated by a permeable, semi-permeable or nanoporous membrane 608 thereby allowing for interaction or diffusion between the respective media flowing through the channels within each compartment. The flow 610 over the cardiomyocyte cells is kept at an appropriate speed in order to reduce sheer stress and provide appropriate oxygenation and sustenance/nutrition to the cells. The second flow 612 (i.e., vascular flow) mimics vascular circulation within the first compartment 601. By providing the cardiomyocytes 606 with a medium specific to the cell type, the cardiomyocytes can receive medium that fulfills the cells' high oxygen demand and specific ionic content requirements. The vascular flow 612 can contain drugs under test as well as metabolites of the drugs produced by other organs which diffuse across the nanoporous membrane 608. Thus, by providing two fluidic channels, the cardiomyocytes 606 may thrive from having access to both the cell-specific medium and can respond to drug challenge to the system. In one embodiment, two or more cardiac cell layers are grown in a three-dimensional manner. The thickness of the tissue layer is limited only by the ability of nutrients to diffuse to the cells from the medium.

B. Lung

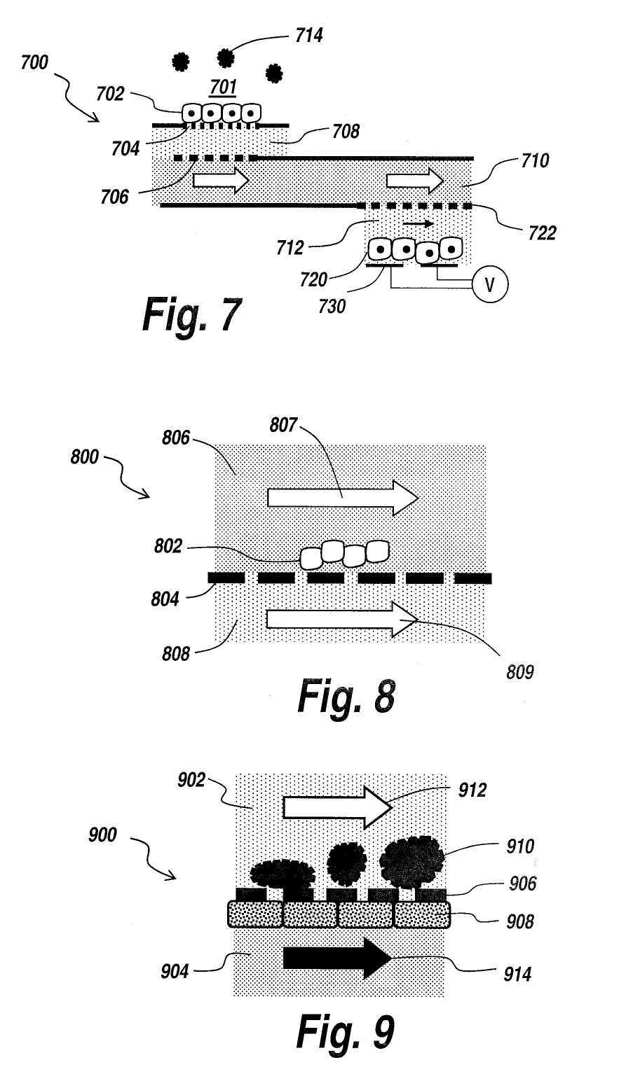

According to one embodiment, the system of the present invention includes a lung module 700, which can be integrated with the heart module noted above. Airway or alveolar cell culture grown at an air liquid interface (ALI) can be used to simulate lung tissue. As shown in FIG. 7, an airway or alveolar cell culture 702 is grown on a first membrane 704 that separates a top compartment 701 from a second or middle compartment 708, and the first membrane defines the air liquid interface. A second membrane 706 separates the middle compartment 708 from a third compartment 710, which serves as a vascular flow compartment. After an early period of submerged growth for the cells 702, the top compartment 701 includes a flow of air therethrough. The second or middle compartment 708 enables flow of the ALI specific tissue medium. The horizontal arrows in FIG. 7 show the flow of blood material through the third compartment 710. The second membrane 706 establishes the interaction by diffusion between the tissue specific medium and the vascular medium. The lung epithelium cells 702 can be exposed to xenobiotics such as nanoparticles 714, but also to pulmonary-delivered pharmaceuticals, respiratory pathogens and toxicants.

The lung construct can be in fluidic connection with a heart module as described above (see FIG. 6). As shown in FIG. 7, a heart module can be integrated with the lung module 700 by coupling an additional compartment 712 containing cardiomyocytes 720 to the vascular compartment 710, with a membrane 722 positioned between the two compartments. A microelectrode array 730 is embedded in the bottom wall of a compartment 712. Operation of the heart module can be conducted as described above in connection with FIG. 6.

Thus, cardiotoxicity of inhaled compounds or particles can be modeled in the system to closely mimic the in vivo physiology. The lung organ mimic can be designed to include more than one cell type to more closely emulate the interface of lung with vasculature. In one embodiment, the lung emulating cell construct can be developed according to the model system as set forth in U.S. Provisional Application No. 61/566,758, the disclosure of which is herein incorporated by reference in its entirety.

C. Liver

According to one embodiment, the system of the present invention includes a liver module. The liver has a central role in drug metabolism and toxicity. Drug-induced liver toxicity is a leading cause of drug failure and the organ is a primary target of chemical and environmental toxicants. The liver also plays a major role in carbohydrate metabolism by removing glucose from the blood, under the influence of the hormone insulin, and storing glucose as glycogen. When the level of glucose in the blood falls, the hormone glucagon causes the liver to break down glycogen and release glucose into the blood. The liver also plays an important role in protein metabolism, primarily through deamination of amino acids, as well as the conversion of the resulting toxic ammonia into urea, which can be excreted by the kidneys. In addition, the liver participates in lipid metabolism by storing triglycerides, breaking down fatty acids, and synthesizing lipoproteins. The liver also secretes bile, which helps in the digestion of fats, cholesterol, phospholipids, and lipoproteins. Analysis of metabolic function will indicate toxicity in liver.

Because of the liver's unique metabolism and relationship to the gastrointestinal tract, the liver is an important target of the toxicity of drugs, xenobiotics, and oxidative stress. Toxicity is often a consequence of the unique vascular, secretory, synthetic, and metabolic features of the liver. About 75% of hepatic blood comes directly from the gastrointestinal viscera and spleen via the portal vein, bringing drugs and xenobiotics absorbed by the gut directly to the liver in concentrated form. Drug-metabolizing enzymes detoxify many xenobiotics but activate the toxicity of others. Injury mechanisms can be a consequence of metabolism and/or direct cell toxicity of chemicals. These mechanisms include bile acid-induced liver cell injury during cholestasis, pathophysiological effects of mitochondrial dysfunction, and cell damage by reactive oxygen and nitrogen species. Mechanisms also include the vascular (Kupffer cells, neutrophils) and intracellular generation of reactive oxygen by mitochondria and xenobiotic-inducible enzymes (e.g. CYP 4502E1). Liver toxicity can also be mediated via an immunological cascade. Biomarkers of liver toxicity include increases in the levels of the liver enzymes alanine aminotransferase (ALT) and aspartate aminotransferase (AST) in serum, and increased bilirubin levels. Specific histo-pathological patterns of liver injury from drug-induced damage include zonal necrosis, hepatitis, cholestasis, steatosis, granulomas, vascular lesions, and neoplasm. (H. Jaeschke, et al., Mechanisms of Hepatotoxicity, Toxicological Sciences 65, 166-176, 2002. V. J. Navarro and J. R. Senior, Drug-Related Hepatotoxicity. N Engl J Med 354:7, 731-739, 2006.)

As illustrated in the liver module 800 of FIG. 8, liver cells 802 may be cultured over a membrane 804 thereby enabling solute exchange between a first compartment 806 and second compartment 808. The flow over the cells 802, indicated by an arrow 807, is kept at an appropriate speed in order to reduce sheer stress and provide appropriate oxygenation and sustenance/nutrition to the cells 802. The second flow (i.e., vascular flow), indicated by an arrow 809, mimics vascular circulation. Secretions from the cell culture, including metabolites and other byproducts of metabolism, can be transported across the membrane 804 into the common blood medium. By providing the necessary flows of culture medium and blood material to the respective compartments, drugs, chemicals or metabolites, as well as mediators of pharmacological or toxicological response (e.g., growth factors or inflammatory mediators such as cytokines) may be transferred between the first compartment 806 and second compartment 808.

In one embodiment, a toxicant, drug or other xenobiotic may be added to the vascular medium and to hepatocyte-specific stress markers and metabolic functions can be measured in the vascular and hepatocyte specific medium. In another embodiment, drugs can be assayed for their ability to reduce or prevent the progress of hepatic necrocytosis and/or accelerate hepatic regeneration. In yet another embodiment, drugs can be screened for efficacy in the treatment of hepatitis viral infections, nucleoside analog antivirals, immunomodulators, immunostimulators (e.g., interferons and other cytokines) or other immune system-affecting drug candidates, including, but not limited to, thymic peptides, isoprinosine, steroids, and Schiff base-forming salicylaldehyde derivatives.

D. Kidney

The system of the present invention can also include a kidney module. The kidney includes an intricate vascular supply and a variety of different cell types, which perform the functions of filtration, re-absorption and excretion. The basic functional unit of the kidney, the nephron, is composed of a vascular filter, the glomerulus, and a resorptive unit, the tubule. The tubular epithelium of the kidney is responsible for re-absorption of water, salts, and various organic compounds. Many transport processes in the kidney are known to be regulated by fluid flow and shear stress. Integration of an in vitro kidney module along with other fluidic organ modules in the system of the present invention allows for a variety of drug studies, disease models, and toxicology studies, including studies focused on the impact of a drug candidate on various kidney cells.

Similar to the liver construct of FIG. 8, the kidney module can include two compartments that are separated by a permeable, semi-permeable or nanoporous membrane thereby allowing for interaction or diffusion between the two compartments. In one embodiment, renal epithelial cells can be cultured and grown on the semi-permeable membrane. The media flow rate over the cells can be adjusted to an appropriate speed, in order to mimic shear stress (e.g., between typically about 0.5 and 5 dyn/cm²) experienced by the respective kidney cells. The medium flowed over the cells can also provide appropriate oxygenation and sustenance/nutrition to the kidney cells or mimic the flow of urine. The medium flowed over the cells can include buffer solution, blood components, whole blood, urine, dialysate, water, or a filtrate there. The second flow (i.e., vascular flow) mimics vascular circulation. In one embodiment, two or more kidney cell types are grown in a three-dimensional manner. An exemplary kidney module can be constructed substantially as shown in FIG. 8 with the liver cells 802 replaced with kidney cells.

According to one embodiment, drugs that are nephrotoxic can be screened. According to one embodiment of the kidney module, drugs can be screened for efficacy in kidney cells when the system include construct comprising kidney cells affected with diseases.

E. Bone Marrow

The system of the present invention may also include a bone marrow module. Similar to the liver construct of FIG. 8, two compartments are separated by a permeable, semi-permeable, or nanoporous membrane thereby allowing for interaction or diffusion between the compartments. Bone marrow cells can be cultured and grown on the membrane in the functional compartment. The media flow rate over the cells can be adjusted to an appropriate speed in order to mimic sheer stress experienced by the respective bone marrow cells. The medium flowed over the cells can also provide appropriate oxygenation and sustenance/nutrition to the bone marrow cells. The second flow (i.e., vascular flow) mimics vascular circulation. In one embodiment, two or more bone marrow cell types can be grown in a three-dimensional manner. An exemplary bone marrow module can be constructed substantially as shown in FIG. 8 with the liver cells 802 replaced with bone marrow cells.

Integration of an in vitro bone marrow module along with other fluidic organ modules in the system of the present invention allows for a variety of drug studies, disease models, and toxicology studies, including studies focused on the impact of a drug candidate on various bone marrow cell diseases such as malignancies, anemias, or leukemias. In one embodiment, bone marrow stroma cells, mesenchymal stem cells, hematopoietic progenitor cells or other bone marrow cells involved in the production of lymphocytes or prevention of lymph backflow are utilized. In one embodiment, the toxic effect of a drug in bone marrow modules of the invention can be detected by monitoring the effect of the agent on stem cell production. Stem cell production can be monitored by methods well known in the art, such as FACS analysis. Stem cells to be monitored include, but are not limited to hematopoietic progenitors, lymphoid progenitors and myeloid progenitors. In addition, the toxic effect of a drug in bone marrow modules can be detected by screening for the development of adverse secondary effects, such as B12 deficiency, pernicious anemia and maturation arrest (failure to divide). Bone marrow modules of the invention can also be used as an indicator system for the development of autoimmune responses. Adverse autoimmune responses will result in the production of antibodies against albumin-drug conjugates. Suspected adverse autoimmune responses in patients could be confirmed by assaying for the undesired albumin-drug conjugates in bone marrow modules of the invention. In one embodiment, drugs can be screened for their ability to increase or decrease production of specific stem cell progenitors, and the differentiated progeny thereof, including, but not limited to erythrocytes, platelets, neutrophils, T cells, B cells, eosinophils, basophils, neutrophils, and monocytes. In an alternative embodiment, drugs can be screened for their ability to improve the function of sub-optimal marrow. For example, improvement in bone marrow proliferation can be monitored by cell counting methods known in the art.

F. Blood-Brain Barrier

The system of the present invention can also include a blood-brain barrier (BBB) module. In one embodiment, inclusion of a BBB module enables testing a drug or delivery vehicle's ability to permeate the blood-brain barrier. Neurotoxicity is a major adverse effect of new drugs, and often implies that a drug or drug metabolite is able to permeate the brain capillary endothelium and affect the central nervous system. Also, many new therapeutic compounds have been developed that target malignancies and other disorders of the brain. Delivering these compounds to diseased tissue remains a difficult challenge, often requiring local drug delivery in the brain by direct infusion of the compounds through a catheter into the brain parenchyma. Integration of an in vitro BBB module along with other fluidic organ modules in the system of the present invention allows for a variety of drug studies, disease models, and toxicology studies, including studies focused on the impact of a drug candidate on brain cells.

The brain module can include two compartments separated by a permeable, semi-permeable or nanoporous membrane, thereby allowing for interaction or diffusion between the respective compartments. The blood-brain barrier is a tight, selective barrier formed by the endothelial cells that line the cerebral capillaries, in close association with perivascular cells such as glial cells and neurons. Given the dynamic interaction between endothelial cells and perivascular cells, sophisticated in vitro models of the BBB include a co-culture of brain endothelial cells interacting via a porous support with glial cells (astrocytes, oligodendrocytes or microglial cells) (Cecchelli et al, Modeling of the blood-brain barrier in drug discovery and development, Nature Review Drug Discovery 6 (2007)).

FIG. 9 illustrates an exemplary co-culture implemented in a BBB module 900 of the present invention. The module 900 consists of a first fluidic chamber 902 and a second fluidic chamber 904 separated by a porous membrane 906. The brain endothelial cells 908 are seeded on the membrane 906 in the vascular compartment 904. Glial cells 910 are cultured on the opposite side of the membrane 906. The media flow (indicated by arrow 912) and the associated rate over the glial cells is adjusted to an appropriate speed and provides appropriate oxygenation and sustenance/nutrition with limited shear stress. The vascular flow (indicated by arrow 914) satisfies the critical feature of a physiological value of flow-induced shear stress on the endothelial cells. Shear stress on the order of typically about 4 dyn/cm² is known to be an essential element to the formation of appropriately tight junctions in the BBB barrier so that the BBB model exhibits in vitro drug permeability values similar to those measured in vivo.

The BBB model of this invention enables not only investigation of the access to the central nervous system of a drug delivered via the vascular circulation, but also enables reproduction of the pharmacokinetic effects of the BBB to systemic circulation of a drug. The pharmacokinetic properties of the BBB, most importantly the BBB's drug metabolism functionality and drug sequestration properties, can affect a drug circulating in the vascular flow pathway of the system of the invention and interacting with all the other organs of the system. Glial cells serve many important support functions in the nervous system. Efficacy or toxicity to glial cells can be evaluated for those agents that pass through the BBB, which may include drug compounds, metabolites, and cell-to-cell signaling molecules.

G. Other Organs

The system may also include other organ modules of an animal or human body. Other organ mimics can be realized using configurations similar to one of those illustrated in FIGS. 6-9 according to the most appropriate emulation of in vivo physiology. The organ mimic parameters such as size and relative vascular flow can be adjusted to be a pharmacokinetically accurate representation of in vivo physiology. Examples of other organ mimics include the skin, gut, adipose tissue, skeletal or smooth muscle, reproductive organs, and the immune system.

### Methods of Use

In use, the system of the present invention can be used to analyze the response of a mammalian or human body to an agent. An end user can administer an agent to one or more cells of at least one organ tissue module or to the blood medium, and evaluate any physiological response or injury to that organ module and all other organ modules in the system. In one embodiment, the agent is a drug, toxin, or pathogen. In one particular embodiment, the system of the present invention can be used to evaluate the efficacy and/or safety of a drug. In one embodiment, diseased cells can be utilized in one or more organ modules to enable testing of efficacy of the drug. In another embodiment, population-specific or patient-specific cells can be used, for example to represent populations with elevated risk of toxicity such as early developmental life stages. The system of the present invention also provides the capability to independently challenge and sample the air and vascular chambers to model inhalation exposure and physiological responses involving blood-borne solute/element recruitment.

The system of the present invention enables analysis of the effluent from cell culture functional compartments of the modules and access to the common vascular fluid. Analysis of cell secretions can be performed with methods known to those skilled in the art (e.g., ELISA or mass spectrometry). The modules can also be analyzed by optical imaging, possibly when disconnected from the fluidic backplane. Access to perfusion and optical imaging enables the user to conduct colorimetric and fluorescent interrogation of cellular functions, including immunostaining.

Although specific embodiments of the present invention are herein illustrated and described in detail, the invention is not limited thereto. The above detailed descriptions are provided as exemplary of the present invention and should not be construed as constituting any limitation of the invention. Modifications will be obvious to those skilled in the art, and all modifications that do not depart from the spirit of the invention are intended to be included with the scope of the appended claims.

1. A microfluidic system comprising:
at least two organ tissue modules, each organ tissue module comprising
a first microfluidic chamber comprising at least one organ tissue cell, wherein the first chamber comprises at least one inlet and at least one outlet for flow of an organ tissue cell-specific culture medium;
a second microfluidic chamber, wherein the second chamber comprises at least one inlet and at least one outlet for flow of a blood material; and
a semi-permeable membrane separating the first and second chamber, wherein the flow of blood material through each organ tissue module is interconnected such that the blood material circulates through a plurality of organ tissue modules within the microfluidic system, and wherein the flow of tissue cell-specific culture medium to each organ tissue module is separated from the flow of tissue cell-specific culture medium to at least one other organ tissue module.

2. The microfluidic system of claim 1, wherein the membrane enables interaction or diffusion between the first and second chamber of each organ tissue module.

3. The microfluidic system of claim 1, wherein the semi-permeable membrane comprises a nanoporous polymer.

4. The microfluidic system of claim 1, wherein each organ tissue module includes a pump operably positioned to move the tissue cell-specific culture medium through the first chamber.

5. The microfluidic system of claim 4, further comprising a pneumatic backplane pneumatically connected to each organ tissue module and including air channels connected to a source of vacuum and a source of positive air pressure for pneumatic operation of at least one medium pump.

6. The microfluidic system of claim 5, wherein the pneumatic backplane includes at least one electrical cable to enable electrical read-out of cellular activity.

7. The microfluidic system of claim 1, wherein each organ tissue module is in fluid communication with a fluidic backplane, wherein the fluidic backplane includes at least one channel for blood material flow, at least one blood material pump, and a reservoir for blood material to enable blood material flow to each tissue module.

8. The microfluidic system of claim 1, wherein the system is adapted to continuously re-circulate blood material through a common fluidic circuit that connects each organ tissue module.

9. The microfluidic system of claim 1, wherein the blood material includes whole blood or a composition comprising a component of whole blood including plasma, proteins, platelets or red blood cells, or an oxygen-carrying blood substitute including hemoglobin-based oxygen carriers, crosslinked and polymerized hemoglobin, and perfluorocarbon-based oxygen carriers.

10. The microfluidic system of claim 1, wherein the blood material flow through each organ tissue module is adapted to pharmacokinetically mimic blood flow in a human.

11. The microfluidic system of claim 1, wherein the system comprises two or more organ tissue modules comprising organ tissue derived from a liver, kidney, bone marrow, heart, brain or blood-brain barrier, or lung.

12. The microfluidic system of claim 1, wherein at least one of organ tissue module size, residence time of cell culture media or blood material in each organ tissue module, and flow distribution of blood material through the microfluidic system are selected based on physiologically based pharmacokinetics.

13. The microfluidic system of claim 1, wherein the at least one organ tissue cell is a primary cell.

14. The microfluidic system of claim 1, wherein the at least one organ tissue cell is located at an air-liquid interface.

15. The microfluidic system of claim 1, wherein the at least one organ tissue cell is located on the semi-permeable membrane or on an interior surface of the first chamber.

16. The microfluidic system of claim 1, wherein the at least one organ tissue cell is part of a co-culture of multiple cell types, wherein all cell types are positioned on one side of the semi-permeable membrane or different cell types are positioned on each side of the semi-permeable membrane.

17. The microfluidic system of claim 1, wherein the at least one organ tissue cell is a three dimensional cell construct.

18. The microfluidic system of claim 1, wherein one of the organ tissue modules is adapted to mimic the heart, and wherein the organ tissue module comprises a first microfluidic chamber comprising a plurality of cardiomyocytes therein and a second microfluidic chamber adapted to receive the flow of a blood material, the first microfluidic chamber separated from the second microfluidic chamber by the semi-permeable membrane, and further comprising a microelectrode array operatively positioned to make electrophysiological measurements of the cardiomyocytes.

19. The microfluidic system of claim 18, wherein the organ tissue module adapted to mimic the heart is an integrated heart/lung organ tissue module further comprising a multi-chamber module adapted to mimic the air-liquid interface of a lung adjacent to the second microfluidic chamber and separated therefrom by a second semi-permeable membrane, the multi-chamber module comprising a first lung chamber adapted to receive a liquid culture medium and positioned adjacent to the second semi-permeable membrane and a second lung chamber comprising alveolar epithelial cells and adapted to receive a flow of air, the second lung chamber separated form the first lung chamber by a third semi-permeable membrane.

20. The microfluidic system of claim 1, wherein one or more organ tissue modules are adapted to mimic an organ selected from the group consisting of liver, kidney, and bone marrow, the organ tissue module comprising a first microfluidic chamber comprising a plurality of cells selected from the group consisting of liver cells, kidney cells, and bone marrow cells, and a second microfluidic chamber adapted to receive the flow of a blood material, the first microfluidic chamber separated from the second microfluidic chamber by the semi-permeable membrane.

21. The microfluidic system of claim 20, wherein the system comprises an organic tissue module comprising liver cells, an organ tissue module comprising kidney cells, and an organ tissue module comprising bone marrow cells.

22. The microfluidic system of claim 1, wherein one of the organ tissue modules is adapted to mimic the blood-brain barrier, and wherein the organ tissue module comprises a first microfluidic chamber comprising a plurality of brain glial cells and a second microfluidic chamber adapted to receive the flow of a blood material and comprising a plurality of brain endothelial cells, the first microfluidic chamber separated from the second microfluidic chamber by the semi-permeable membrane.

23. The microfluidic system of claim 22, wherein the brain glial cells and the brain endothelial cells are seeded on opposite sides of the semi-permeable membrane.

24. The microfluidic system of claim 1, wherein each organ tissue module is in fluid communication with a fluidic backplane that defines a recirculating flow path for the flow of blood material such that the blood material can continuously circulate through each second microfluidic chamber of each organ tissue module, and wherein each organ tissue module is removably connected to the fluidic backplane.

25. The microfluidic system of claim 24, wherein each organ tissue module defines a separate flow path for the organ tissue cell-specific culture medium that is wholly contained within the organ tissue module.

26. The microfluidic system of claim 25, wherein the flow path for the organ tissue cell-specific culture medium comprises a channel extending from a first reservoir on the organ tissue module, through the first microfluidic chamber, and to a second reservoir on the organ tissue module.

27. The microfluidic system of claim 26, wherein each organ tissue module includes a pump operably positioned to move the organ tissue cell-specific culture medium through the first microfluidic chamber.

28. The microfluidic system of claim 27, further comprising a pneumatic backplane pneumatically connected to each organ tissue module and adapted for pneumatic operation of the pump.

29. The microfluidic system of claim 24, wherein the system comprises the following:
a lung module comprising alveolar epithelial cells and adapted to mimic the air-liquid interface of a lung;
a heart module comprising cardiomyocytes; and
at least one of (i) a blood-brain barrier module comprising brain glial cells and brain endothelial cells; (ii) a liver module comprising liver cells; (iii) a kidney module comprising kidney cells; and (iv) a bone marrow module comprising bone marrow cells;
wherein the blood material flow through each organ tissue module is adapted to pharmacokinetically mimic blood flow in a human.

30. A method of analyzing tissue response to an agent comprising:
providing a microfluidic system according claim 1;
administering an agent to the organ tissue cells of at least one organ tissue module; and
evaluating any physiological response or injury to organ tissue cells contained in any of the organ tissue modules.

31. The method of claim 30, wherein the agent is a drug, toxin or pathogen.

32. The method of claim 30, wherein the administering step comprises administering the agent to the flow of blood material.

33. The method of claim 30, wherein the administering step comprises administering the agent to an organ tissue cell-specific culture medium of one or more of the organ tissue modules.

34. The method of claim 30, wherein the microfluidic system comprises a lung module adapted to mimic the air-liquid interface of the lung, and the administering step comprises administering the agent to the air at the air-liquid interface.

35. The method of claim 30, wherein the evaluating step comprises analysis of cell secretions from one or more organ tissue modules or optical imaging of one or more organ tissue modules.
